(19) United States
(12) Patent Application Publication
(10) Pub. No.: US 2020/0043362 A1
(43) Pub. Date: Feb. 6, 2020

(54) APPARATUS AND METHOD OF OPERATING THE APPARATUS TO GUIDE A USER IN A LONG-TERM MEMORY TRAINING SESSION

(71) Applicant: KONINKLIJKE PHILIPS N.V., EINDHOVEN (NL)

(72) Inventors: Björn Nicolaas Servatius VLASKAMP, EINDHOVEN (NL); Laura KLAMING, AMSTERDAM (NL)

(21) Appl. No.: 16/339,766

(22) PCT Filed: Oct. 5, 2017
(86) PCT No.: PCT/EP2017/075403

§ 371 Date: Apr. 5, 2019

(30) Foreign Application Priority Data
Oct. 5, 2016 (EP) 16192406.3

(51) Int. Cl. G09B 19/00; A61B 5/16; G09B 7/00

(52) U.S. Cl. CPC G09B 19/003; G09B 7/00; A61B 5/165

(57) ABSTRACT

There is provided an apparatus and method of operating the apparatus to guide a user in a long-term memory training session. One or more items are rendered to the user in the long-term memory training session (202). A request is initiated for the user to recall at least one rendered item that directly precedes the request (204). A memory strategy applied by the user to the long-term memory training session is determined based on a response received from the user to the request (206). When the memory strategy applied by the user to the long-term memory training session is determined to be a working memory strategy (208), an action is initiated to guide the user to apply a long-term memory strategy to the long-term memory training session (210).

### FIELD OF THE INVENTION

The invention relates to an apparatus and a method of operating the apparatus to guide a user in a long-term memory training session.

### BACKGROUND OF THE INVENTION

Memory training is known to improve the performance of a user in memory tasks and related activities. This can be particularly beneficial to help users such as young children, elderly people, or people with memory issues. Memory training can be provided in various forms including digital applications that run on mobile devices (such as smart phones, tablets, or laptops), or programs that run on other devices (such as personal computers). One of the components of a memory training application or program is a module that teaches memory strategies.

Examples of memory tasks that can benefit from memory training include everyday tasks, particularly those which rely on episodic memory. For example, everyday tasks can include remembering grocery lists or appointments. However, for memory training to be effective, it is important that the user applies the correct memory strategy during a training session since memory training can target different subcomponents of memory. For example, some memory training is targeted at the improvement of working memory, whereas other memory training is aimed at improving components of long-term memory (such as episodic memory). If the user relies on components of memory other than the component of memory targeted by the memory training, the memory training would be ineffective.

This can happen, in particular, in episodic and/or semantic memory training where there is a relatively short time interval (for example, seconds or minutes) between presentation of information and recall of that information. In these cases, the user may rely on their working memory instead of using a strategy to transfer information to their long-term memory. This can happen when the user does not understand the instructions of the memory strategy taught to him prior to a memory training session or because, in the short term, using working memory rather than the taught memory strategy require less effort from the user. Also, information is retained in working memory as long as it is actively processed (e.g. by rehearsing the information) or until new information interferes with the information in working memory leading to the loss of that information. A way to circumvent using long-term (including episodic/semantic) memory is by maintaining information in working memory until asked to recall the information.

When teaching a long-term memory strategy, it is therefore important to ensure that the user relies on their long-term memory and not on their working memory. The working memory is a completely separate part of the memory system to the long-term memory, both functionally and anatomically. It would thus be useful to detect when working memory is being used in a long-term memory training session to guide the user to use the correct (long-term memory) strategy. However, it is not possible to observe directly from the behaviour of the user whether the user relied on their working memory or whether the user applied the long-term memory strategy.

There is thus a need for an apparatus and method for guiding a user in a long-term memory training session. There is also a need for a more efficient and effective long-term memory training such that the training can be completed faster without repetition to relieve the burden on the user and conserve processing and/or energy resources (for example, to reduce the need for battery recharge when the method is run on a battery powered device).

### SUMMARY OF THE INVENTION

As noted above, a limitation with existing techniques is that it is not possible to observe whether the working memory of the user is being used directly from the behaviour of the user. It would thus be beneficial to guide the user to use a long-term memory strategy in a long-term memory training session. It would also be beneficial to enable a more efficient and effective long-term memory training such that the training can be completed faster without repetition to relieve the burden on the user and conserve processing and/or energy resources (for example, to reduce the need for battery recharge when the method is run on a battery powered device). Thus, an improved training is needed and, more specifically, an improved apparatus and method that overcomes the existing technical problems described earlier.

Therefore, according to a first aspect, there is provided a method of operating an apparatus to guide a user in a long-term memory training session. The method comprises rendering one or more items to the user in the long-term memory training session, initiating a request for the user to recall at least one rendered item that directly precedes the request, determining a memory strategy applied by the user to the long-term memory training session based on a response received from the user to the request, and initiating an action to guide the user to apply a long-term memory strategy to the long-term memory training session when the memory strategy applied by the user to the long-term memory training session is determined to be a working memory strategy.

In some embodiments, a request for the user to recall at least one rendered item that directly precedes the request may be initiated at irregular or regular time intervals.

In some embodiments, the memory strategy applied by the user to the long-term memory training session may be determined to be a working memory strategy where the response received from the user to the request comprises an incorrect recall of the at least one rendered item that directly precedes the request.

In some embodiments, the memory strategy applied by the user to the long-term memory training session may be determined to be a long-term memory strategy where the response received from the user to the request comprises a correct recall of the at least one rendered item that directly precedes the request.

In some embodiments, the method may further comprise continuing the long-term memory training session when the memory strategy applied by the user to the long-term memory training session is determined to be a long-term memory strategy.

In some embodiments, initiating an action may comprise adapting the long-term memory training session to guide the user to apply a long-term memory strategy.

In some embodiments, adapting the long-term memory training session may comprise any one or more of: initiating a notification associated with a long-term memory strategy to the user to guide the user to apply a long-term memory strategy, adding at least one item to the one or more items rendered to the user to guide the user to apply a long-term memory strategy, rendering a negative result associated with the working memory strategy to the user to guide the user to apply a long-term memory strategy, and allowing the user to re-start the long-term memory training session to guide the user to apply a long-term memory strategy.

In some embodiments, two or more items may be rendered in a predefined series.

In some embodiments, at least seven items may be rendered to the user in the long-term memory training session and the request for the user to recall at least one rendered item that directly precedes the request may be initiated after rendering seven items to the user.

According to a second aspect, there is provided a computer program product comprising a computer readable medium, the computer readable medium having computer readable code embodied therein, the computer readable code being configured such that, on execution by a suitable computer or processor, the computer or processor is caused to perform the method or the methods described above.

According to a third aspect, there is provided an apparatus for guiding a user in a long-term memory training session. The apparatus comprises a control unit configured to control a user interface to render one or more items to the user in the long-term memory training session, control the user interface to initiate a request for the user to recall at least one rendered item that directly precedes the request, determine a memory strategy applied by the user to the long-term memory training session based on a response received from the user to the request, and initiate an action to guide the user to apply a long-term memory strategy to the long-term memory training session when the memory strategy applied by the user to the long-term memory training session is determined to be a working memory strategy.

In some embodiments, the apparatus may further comprise the user interface.

In some embodiments, the apparatus may further comprise a memory unit for storing one or more actions to guide the user to apply a long-term memory strategy to the long-term memory training session.

In some embodiments, the control unit may be configured to acquire at least one action from the memory unit to initiate to guide the user to apply a long-term memory strategy to the long-term memory training session.

There is also provided a method of operating an apparatus comprising a control unit to guide a user in a long-term memory training session provided by a user interface. The method comprises controlling, by the control unit, the user interface to render one or more items to the user in the long-term memory training session and controlling, by the control unit, the user interface to initiate a request for the user to recall at least one rendered item that directly precedes the request. The method also comprises selecting, by the control unit from a plurality of memory strategies stored in a memory unit, a memory strategy applied by the user to the long-term memory training session provided by the user interface based on a response received from the user to the request and initiating, by the control unit, an action to cause the user interface to guide the user to apply a long-term memory strategy to the long-term memory training session provided by the user interface in the event that the memory strategy applied by the user to the long-term memory training session provided by the user interface is selected to be a working memory strategy.

In some embodiments, the control unit may control the user interface to initiate the request for the user to recall at least one rendered item that directly precedes the request at irregular or regular time intervals.

In some embodiments, the memory strategy applied by the user to the long-term memory training session provided by the user interface may be selected, by the control unit, to be a working memory strategy in the event that the response received from the user to the request comprises an incorrect recall of the at least one rendered item that directly precedes the request. In some embodiments, the memory strategy applied by the user to the long-term memory training session provided by the user interface may be selected, by the control unit, to be a long-term memory strategy in the event that the response received from the user to the request comprises a correct recall of the at least one rendered item that directly precedes the request.

In some embodiments, the method may further comprise continuing the long-term memory training session provided by the user interface when the memory strategy applied by the user to the long-term memory training session provided by the user interface is selected, by the control unit, to be a long-term memory strategy.

In some embodiments, initiating, by the control unit, an action to cause the user interface to guide the user to apply a long-term memory strategy may comprise adapting, by the control unit, the long-term memory training session provided by the user interface to guide the user to apply a long-term memory strategy. In some embodiments, adapting, by the control unit, the long-term memory training session provided by the user interface may comprise any one or more of: controlling, by the control unit, the user interface to initiate a notification associated with a long-term memory strategy to the user to guide the user to apply a long-term memory strategy; adding, by the control unit, at least one item to the one or more items rendered by the user interface to the user to guide the user to apply a long-term memory strategy; controlling, by the control unit, the user interface to render a negative result associated with the working memory strategy to the user to guide the user to apply a long-term memory strategy; and controlling, by the control unit, the user interface to instruct the user to re-start the long-term memory training session provided by the user interface to guide the user to apply a long-term memory strategy.

In some embodiments, the control unit may control the user interface to render two or more items in a predefined series. In some embodiments, the control unit may control the user interface to render at least seven items to the user in the long-term memory training session provided by the user interface and the control unit controls the user interface to initiate the request for the user to recall at least one rendered item that directly precedes the request after rendering seven items to the user.

There is also provided a computer program product comprising a computer readable medium, the computer readable medium having computer readable code embodied therein, the computer readable code being configured such that, on execution by a suitable computer or processor, the computer or processor is caused to perform the method or the methods described above.

There is also provided an apparatus for guiding a user in a long-term memory training session provided by a user interface. The apparatus comprises a control unit configured to control the user interface to render one or more items to the user in the long-term memory training session provided by the user interface and control the user interface to initiate a request for the user to recall at least one rendered item that directly precedes the request. The control unit is also configured to select, from a plurality of memory strategies stored in a memory unit, a memory strategy applied by the user to the long-term memory training session provided by the user interface based on a response received from the user to the request and initiate an action to cause the user interface to guide the user to apply a long-term memory strategy to the long-term memory training session provided by the user interface in the event that the memory strategy applied by the user to the long-term memory training session provided by the user interface is selected to be a working memory strategy.

In some embodiments, the apparatus may further comprise the user interface. In some embodiments, the apparatus may further comprise a memory unit for storing one or more actions to guide the user to apply a long-term memory strategy to the long-term memory training session provided by the user interface. In some embodiments, the control unit may be configured to acquire at least one action from the memory unit to initiate to guide the user to apply a long-term memory strategy to the long-term memory training session provided by the user interface.

According to the aspects and embodiments described above, the limitations of existing techniques are addressed. For example, it is possible to determine when a user is using a working memory strategy to initiate actions to guide the user to use a long-term memory strategy. In this way, the effectiveness of a long-term memory training session can be improved as well as the user experience and motivation to continue with the memory training. The method and apparatus described above enable a more efficient use of a long-term memory training session, thereby increasing the possibility of the user remembering elements of importance to the user. These elements of importance may, for example, include security related aspects (such as remembering that an oven is on, a door is unlocked, and so on), health related aspects (such as remembering to take medications, attend a medical appointment, acquire measurements for health monitoring or assessment, and so on), and any other elements of importance.

The method and apparatus also provide a more tailored and improved long-term memory training, which results in the training being more effective such that it is not necessary for the user to repeat the training. Also, since a determination is made on the memory strategy that is applied, it is possible to find out at the time of training whether the training will be effective so that the user can be guided to use the long-term memory strategy if they are not already doing so. In this way, the user is able to learn faster, which relieves the burden on the user. This also conserves processing and/or energy resources since the training session can be completed effectively in a shorter time and will less likely need to be repeated. Advantageously, when the method is run on a battery powered device, a more effective training can be provided before battery recharge is needed. The method and apparatus described above thus provides a technical tool to serve and assist a user in a memory training activity, which enables the user to perform a memory training task more efficiently. In this way, an improved training is provided to the user.

There is thus provided an improved apparatus and method for guiding a user in a long-term memory training session, which overcomes the existing problems.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a block diagram of an apparatus according to an embodiment;

FIG. 2 is a flow chart illustrating a method of operating an apparatus to guide a user in a long-term memory training session according to an embodiment; and

FIG. 3 is a block diagram illustrating an example method of rendering one or more items and requests according to an embodiment.

### DETAILED DESCRIPTION OF EMBODIMENTS

As noted above, the invention provides an improved apparatus and method for guiding a user in a long-term memory training session, which overcomes the existing problems.

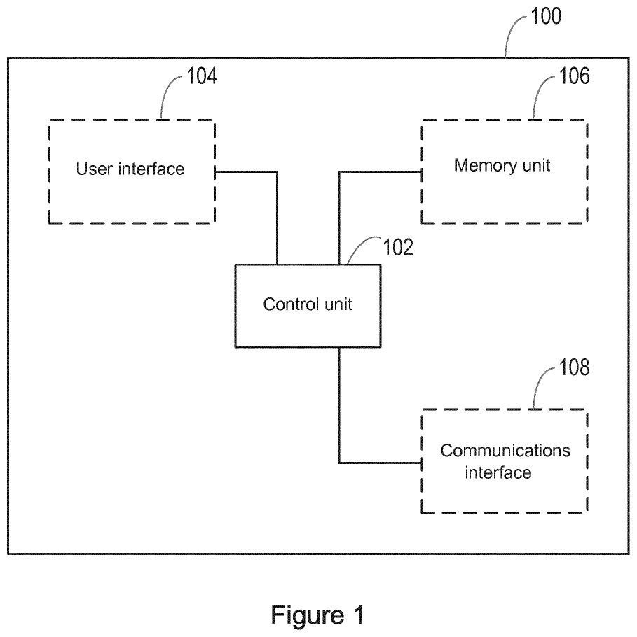

FIG. 1 shows a block diagram of an apparatus 100 according to an embodiment that can be used for guiding a user in a long-term memory training session (or task or activity).

The apparatus 100 comprises a control unit 102 that controls the operation of the apparatus 100 and that can implement the method described herein. The control unit 102 can comprise one or more processors, control units, multi-core processors or modules that are configured or programmed to control the apparatus 100 in the manner described herein. In particular implementations, the control unit 102 can comprise a plurality of software and/or hardware modules that are each configured to perform, or are for performing, individual or multiple steps of the method according to embodiments of the invention.

Briefly, the control unit 102 of the apparatus 100 is configured to control a user interface to render one or more items to the user in the long-term memory training session, control the user interface to initiate a request for the user to recall a rendered item that directly precedes the request, determine (or ascertain, establish, deduce, detect, decide on, or select) a memory strategy applied by the user to the long-term memory training session based on a response received from the user to the request, and initiate an action to guide the user to apply a long-term memory strategy to the long-term memory training session when the memory strategy applied by the user to the long-term memory training session is determined to be a working memory strategy.

A long-term memory is a capacity of the user to retain information (such as items) over a long or extended period of time. For example, the period of time a user can store information in a long-term memory may be any time period and can be of the order of minutes, hours, days, months or years. Thus, a user that applies a long-term memory strategy is likely to retain an item in memory for more than a few seconds. After this time, the user is still likely to be able to correctly recall the item. Generally, any information that is actively processed by a user is transferred to (or encoded in) the long-term memory of the user. Any information that is not actively processed by the user will be lost after a short or limited period of time if it remains unprocessed.

Working memory is a capacity of the user to retain, manipulate and process information (such as items) over a short or limited period of time. The capacity of a working memory is, on average, seven items or around seven items (for example, between five to nine items, i.e. five items, six items, seven items, eight items, or nine items). Once the working memory of a user is fully occupied, the user is not able to store further items in their working memory unless the user processes the already stored items such that the items are transferred into their long-term memory. Also, information can only be held in working memory for a short or limited period of time, which is of the order of seconds (such as a few seconds). When the working memory of a user is fully occupied, for example, by the user applying a working memory strategy (such as rehearsal), no more information can be taken up or retained in working memory. As a consequence, the user will not be able to correctly recall an item that has not yet been transferred to long-term memory.

Returning back to the illustrated embodiment of FIG. 1, in this illustrated embodiment, the apparatus 100 comprises the user interface 104 that is controlled by the control unit 102. Alternatively, the user interface 104 may be external to (i.e. separate to or remote from) the apparatus 100. For example, the user interface 104 may be part of another device. The user interface 104 is for use in rendering one or more items to the user in the long-term memory training session and rendering a request initiated by the control unit 102 for the user to recall a rendered item that directly precedes the request.

The user interface 104 may also be for use in providing the user of the apparatus 100 with information resulting from the method according to the invention. The control unit 102 may be configured to control the user interface 104 to provide information resulting from the method according to the invention. For example, in some embodiments, the control unit 102 of the apparatus 100 may be configured to control the user interface 104 to render (or output) information associated with one or more actions initiated by the control unit 102 to guide the user to apply a long-term memory strategy to the long-term memory training session. The user interface 104 may also be configured to receive a user input. In other words, a user interface 104 may allow the user to manually enter data, instructions, or information. For example, the user interface 104 may be configured to receive a response from the user to a request for the user to recall a rendered item.

Thus, the user interface 104 may be or may comprise any component that enables rendering or output of information, data or signals to the user. Alternatively or in addition, the user interface 104 may be or may comprise any component that enables the user to provide a user input, interact with and/or control the apparatus 100. For example, the user interface 104 may comprise one or more switches, one or more buttons, a keypad, a keyboard, a touch screen or an application (for example, on a tablet or smartphone), a display screen or any other visual rendering component, one or more lights, one or more speakers or any other audio rendering component, or any other user interface component, or combination of user interface components.

In some embodiments (such as the illustrated embodiment of FIG. 1), the apparatus 100 may comprise a memory unit 106 configured to store program code that can be executed by the control unit 102 to perform the method described herein. The memory unit 106 can also be used to store information, data, signals and measurements made or acquired by the control unit 102 of the apparatus 100 or by components, interfaces, units, and devices that are external to the apparatus 100. Thus, for example, the memory unit 106 of the apparatus 100 may be configured to store one or more actions to guide the user to apply a long-term memory strategy to the long-term memory training session and the control unit 102 may be configured to acquire at least one action from the memory unit 106 to initiate to guide the user to apply a long-term memory strategy to the long-term memory training session. However, it will be understood that the control unit 102 may alternatively or additionally be configured to acquire at least one action from one or more memory units that are external to (i.e. separate to or remote from) the apparatus 100.

Returning back to FIG. 1, in some embodiments, the apparatus 100 may also comprise a communications interface (or circuitry) 108 for enabling the apparatus 100 to communicate with (or connect to) any components, interfaces, units, and devices that are internal or external to the apparatus 100. The communications interface 108 may communicate with any components, interfaces units, and devices wirelessly or via a wired connection. For example, in embodiments where the user interface 104 is external to the apparatus 100, the communications interface 108 may communicate with the external user interface wirelessly or via a wired connection. Similarly, in embodiments where one or more memory units 106 are external to the apparatus 100, the communications interface 108 may communicate with the external memory units wirelessly or via a wired connection.

It will be appreciated that FIG. 1 only shows the components required to illustrate this aspect of the invention, and in a practical implementation the apparatus 100 may comprise additional components to those shown. For example, the apparatus 100 may comprise a battery or other power supply for powering the apparatus 100 or means for connecting the apparatus 100 to a mains power supply.

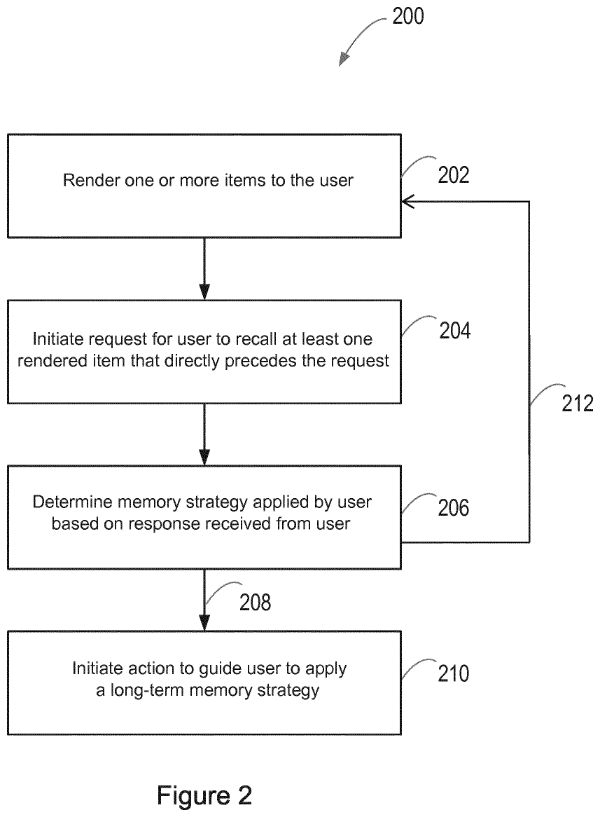

FIG. 2 illustrates a method 200 of operating an apparatus to guide a user in a long-term memory training session (or task or activity) according to an embodiment. The illustrated method 200 can generally be performed by or under the control of the control unit 102 of the apparatus 100.

Generally, the aim of a memory training session is to teach a memory strategy to a user. This can comprise rendering instructions to the user to teach the user a particular memory strategy and the subsequent rendering of items that the user is requested to recall after a predefined time interval using the taught memory strategy, which allows the user to practice the strategy. A long-term memory training session can be any training session in which the user is requested to use a long-term memory strategy. For example, a long-term memory strategy may comprise a visual mnemonic memory strategy, a verbal mnemonic strategy or any other long-term memory strategy. A visual mnemonic strategy is a strategy in which the user makes a mental image of the information to be memorised. A verbal mnemonic strategy is a strategy in which the user uses words to memorise the information. This can, for example, be through the use of rhyming, formulating implementation intentions, or categorising information semantically.

The long-term memory strategy may comprise any memory strategy, e.g. visual memory strategy, implementation intentions, rhyming, semantic categorisations, or any other long-term memory strategy. A long-term memory strategy may be taught to the user prior to the long-term memory training session in which the user is to apply the long-term memory strategy. For example, in some embodiments, the control unit 102 may control the user interface (which may be a user interface 104 of the apparatus 100 or a user interface external to the apparatus 100) to render an explanation of the long-term memory strategy to the user prior to the long-term memory training session.

With reference to FIG. 2, at block 202, one or more items are rendered to the user in the long-term memory training session. The one or more items may include audio items that can be rendered via audio (for example, via an audio rendering component of the user interface such as a speaker), visual items that can be rendered visually (for example, via a visual rendering component of the user interface such as a display screen), or any combination of audio and visual items. In some examples, the items can comprise words, sentences, images, or any other audio and/or visual items. Specifically, the control unit 102 of the apparatus 100 may control the user interface (which may be a user interface 104 of the apparatus 100 or a user interface external to the apparatus 100) to render the one or more items to the user. The aim is for the user to remember the one or more rendered items using a long-term memory strategy. In some embodiments, two or more items may be rendered in a predefined or random series (or sequence or order).

At block 204, a request is initiated for the user to recall at least one rendered item that directly precedes the request. Specifically, the control unit 102 of the apparatus 100 may control the user interface (which may be a user interface 104 of the apparatus 100 or a user interface external to the apparatus 100) to render the initiated request for the user to recall at least one rendered item that directly precedes the request. The initiated request can comprise an audio request (for example, rendered via an audio rendering component of the user interface such as a speaker) or a visual request (for example, rendered via a visual rendering component of the user interface such as a display screen).

In some embodiments, the request may be initiated for the user to recall a rendered item that directly precedes the request (or the item rendered immediately prior to the request). In other embodiments, the request may be initiated for the user to recall more than one rendered item that directly precede the request (or a predefined number of items rendered immediately prior to the request). In an embodiment in which two or more items are rendered in a predefined or random order, the initiated request may be for the user to recall two or more items that directly precede the request in the order in which the items are rendered (for example, one at a time in the order in which the items are rendered). In this embodiment, alternatively or in addition, at least one item that directly precedes the request may be blank (or missing) and the initiated request may comprise a request for the user to recall the item that is blank (or missing) based on the order in which the two or more items are previously rendered.

The request for the user to recall at least one rendered item that directly precedes the request may be a single request that is initiated or may be a request that is initiated at regular or irregular time intervals. For example, the predefined number of items rendered to the user prior to initiating each request may be the same such that the request is initiated at regular intervals, or the predefined number of items rendered to the user prior to initiating each request may vary such that the request is initiated at irregular intervals. In an example embodiment, at least seven items may be rendered to the user in the long-term memory training session and the request for the user to recall a rendered item that directly precedes the request may be initiated after rendering seven items to the user.

Although an example is provided in which the request is initiated after rendering seven items to the user, it will be understood that the request may be imitated after rendering any integer number of items to the user (for example, after rendering one, two, three, four, five, six, seven, eight, nine, ten, eleven, twelve, or any other integer number, of items to the user). In some embodiments, the predefined number of items after which a recall request is initiated may be set based on a working memory capacity of the user. In addition or alternatively, in some embodiments, the predefined number of items after which a recall request is initiated may be set based on the performance of the user in the memory training session. For example, the predefined number of items (and thus the time interval) after which a recall request is initiated may be increased where it is determined that the performance of the user is improving.

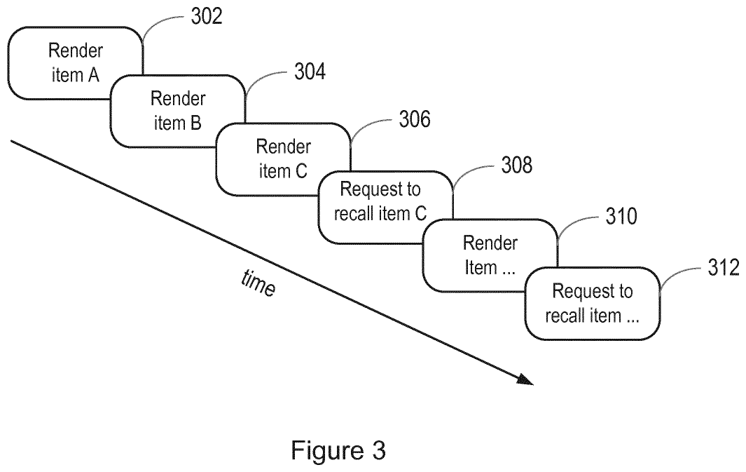

FIG. 3 illustrates an example method of rendering one or more items (block 202 of Figure) and requests (block 204 of FIG. 2) over time according to an embodiment. Specifically, the method of FIG. 3 illustrates an example flow (or order) for the rendering of items and requests according to the embodiment. With reference to FIG. 3, over a predefined time period, a first item A is rendered to the user (at block 302), a second item B is rendered to the user (at block 304), and a third item C is rendered to the user (at block 306). Then, a request for the user to recall the third item C is rendered to the user (at block 308). This request is intended to test whether the user is applying a working memory strategy to memorise the third item C since the request immediately follows the third item C. Following the request, over time, one or more further items are rendered to the user (at block 310) and one or more further recall requests are rendered to the user (at block 312). In other words, one or more items are rendered to the user and, at predefined time intervals, a request for the user to recall the latest item is rendered to the user.

Although examples have been provided for the structure, order, and content of items rendered to the user and requests to recall items, it will be understood that other structures, orders, and content are also possible. For example, the structure, order, and content of items rendered to the user and requests to recall items may depend on the long-term memory strategy that is taught to the user prior to the long-term memory training session.

Returning back to FIG. 2, at block 206, a memory strategy applied by the user to the long-term memory training session is determined (or ascertained, established, deduced, detected, decided on, or select) based on a response received from the user to the request for the user to recall a rendered item that directly precedes the request. For example, the control unit 102 of the apparatus 100 may select (from a plurality of memory strategies stored in a memory unit 106) a memory strategy applied by the user to the long-term memory training session provided by the user interface 104 based on a response received from the user to the request.

Where a response received from the user to the request comprises a correct recall of the rendered item that directly precedes the request, it is determined that the memory strategy applied by the user to the long-term memory training session is not a working memory strategy (for example, it is determined that the working memory of the user is not occupied). If it is determined that the user is not applying a working memory strategy to the long-term memory training session, it is likely that the user is correctly applying a long-term memory strategy to the long-term memory training session. Thus, for example, where the response received from the user to the request comprises a correct recall of the rendered item that directly precedes the request, the memory strategy applied by the user to the long-term memory training session may be determined to be a long-term memory strategy. Generally, if the user is applying a long-term memory strategy to the long-term memory training session, the response received from the user to the request to recall a rendered item that directly precedes the request is likely to be correct since this latest rendered item will still be in the working memory of the user. In this way, it is possible to detect long-term memory usage of the user.

At block 206 of FIG. 2, when the memory strategy applied by the user to the long-term memory training session is determined to be a long-term memory strategy, the long-term memory training session is continued (as indicated by the arrow 212 in FIG. 2). Specifically, the control unit 102 continues to control the user interface (which may be a user interface 104 of the apparatus 100 or a user interface external to the apparatus 100) to render items to the user (block 202 of FIG. 2), continues to initiate requests for the user to recall latest rendered items (block 204 of FIG. 2), and continues to determine a memory strategy applied by the user to the long-term memory training session (block 206 of FIG. 2).

On the other hand, where a response received from the user to the request comprises an incorrect recall of the rendered item that directly precedes the request, the memory strategy applied by the user to the long-term memory training session is determined to be a working memory strategy. If it is determined that the user is applying a working memory strategy to the long-term memory training session, it is determined that the user is applying an incorrect memory strategy in that it is likely that the user is not applying a long-term memory strategy to the long-term memory training session. Generally, if the user is applying a working memory strategy rather than a long-term memory strategy to the long-term memory training session, the working memory of the user is occupied with items rendered prior to the latest rendered item (or other items remembered by rehearsal), leaving no working memory capacity for the latest rendered item. As such, the response received from the user to the request to recall a rendered item that directly precedes the request is likely to be incorrect. In this way, it is possible to detect working memory usage of the user.

As mentioned above, in an example embodiment, the request for the user to recall a rendered item that directly precedes the request may be initiated after rendering seven items to the user. This can be beneficial in determining, in the manner described above, a memory strategy applied by the user to the long-term memory training session since often a working memory can contain a maximum of seven (or around seven) items. Thus, if the memory strategy applied by the user to the long-term memory training session is determined to be a working memory strategy, a response received from the user to a request initiated after seven items is likely to comprise an incorrect recall of the rendered item that directly precedes the request. An error in the recall of a rendered item that directly precedes the request after seven items are rendered can provide a strong indication that a working memory strategy is applied by the user.

At block 206 of FIG. 2, when the memory strategy applied by the user to the long-term memory training session is determined to be a working memory strategy, the method proceeds to block 208 of FIG. 2 (as indicated by the arrow 208). At block 208 of FIG. 2, an action is initiated to guide the user to apply a long-term memory strategy to the long-term memory training session. In some embodiments, the initiation of an action may comprise adapting the long-term memory training session to guide the user to apply a long-term memory strategy. For example, the long-term memory training session may be adapted to ensure that the user learns to apply a long-term memory strategy (which may be a long-term memory strategy taught to the user prior to the long-term memory training session).

The adaptation of the long-term memory training session can comprise initiating to the user a notification associated with a long-term memory strategy to guide the user to apply a long-term memory strategy. Specifically, the control unit 102 of the apparatus 100 may control the user interface (which may be a user interface 104 of the apparatus 100 or a user interface external to the apparatus 100) to render the notification to the user to guide the user to apply a long-term memory strategy. The notification can comprise an audio notification (for example, rendered via an audio rendering component of the user interface such as a speaker) or a visual notification (for example, rendered via a visual rendering component of the user interface such as a display screen).

The notification may comprise any one or more of instructions (or additional instructions where instructions have already been provided) indicating or explaining how the user can apply a long-term memory strategy, an indication that the user is applying an incorrect memory strategy, information instructing the user to look up a previously rendered explanation of the long-term memory strategy (which may, for example, be stored in the memory unit 106 of the apparatus 100 or a memory unit external to the apparatus 100), or any other notification suitable to guide the user to apply a long-term memory strategy. The instructions (or additional instructions where instructions have already been provided) indicating how the user can apply a long-term memory strategy may comprise, for example, information associated with the use of the long-term memory training session, information that can educate the user on the reasons for using a long-term memory strategy, hints and/or tips on how to memorise items, or any other information or combination of information that can guide the user to apply a long-term memory strategy.

Alternatively or in addition, the adaptation of the long-term memory training session can comprise adding at least one item to the one or more items rendered to the user to guide the user to apply a long-term memory strategy. For example, the at least one item added to those rendered to the user may include one or more items intended to compensate for the lack of training of previous items due to the user applying a working memory strategy instead of a long-term memory strategy. In this way, where an incorrect memory strategy is applied to previous items, the user is provided with further items to memorise. This can increase the length of the training session to provide the user with more practice of applying a long-term memory strategy.

Alternatively or in addition, the adaptation of the long-term memory training session can comprise rendering a negative result associated with the working memory strategy to the user to guide the user to apply a long-term memory strategy. For example, in an embodiment, the control unit 102 may be configured to allocate points to the user during the long-term memory training session when the user applies a long-term memory strategy. This can provide the user with an indication of their progress during the long-term memory training session. Thus, a negative result associated with the working memory strategy may comprise reducing the points allocated to the user to guide the user to apply a long-term memory strategy.

Alternatively or in addition, the adaptation of the long-term memory training session can comprise allowing the user to re-start the long-term memory training session to guide the user to apply a long-term memory strategy.

In any of the embodiments described herein, the method or methods may be fully (or at least partially) automated.

Therefore, as described above, there is provided an improved apparatus and method for guiding a user in a long-term memory training session (or task or activity). Specifically, the apparatus and method allow a determination to be made as to whether a user is applying a working memory strategy to a long-term memory training session to initiate actions to guide the user to use the correct long-term memory strategy. In this way, the effectiveness of a memory training session can be improved as well as the user experience and motivation to continue with the memory training. The method and apparatus thus provides a technical tool to serve and assist a user in a memory training activity, which enables the user to perform a memory training task more efficiently.

There is also provided a computer program product comprising a computer readable medium, the computer readable medium having computer readable code embodied therein, the computer readable code being configured such that, on execution by a suitable computer or processor, the computer or processor is caused to perform the method or methods described herein.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method of operating an apparatus comprising a control unit to guide a user in a long-term memory training session provided by a user interface, the method comprising:
controlling, by the control unit, the user interface to render one or more items to the user in the long-term memory training session;
controlling, by the control unit, the user interface (104) to initiate a request for the user to recall at least one rendered item that directly precedes the request;
selecting, by the control unit from a plurality of memory strategies stored in a memory unit, a memory strategy applied by the user to the long-term memory training session provided by the user interface based on a response received from the user to the request; and
initiating, by the control unit, an action to cause the user interface to guide the user to apply a long-term memory strategy to the long-term memory training session provided by the user interface in the event that the memory strategy applied by the user to the long-term memory training session provided by the user interface is selected to be a working memory strategy, wherein initiating, by the control unit, an action to cause the user interface to guide the user to apply a long-term memory strategy comprises:
adapting, by the control unit, the long-term memory training session provided by the user interface to guide the user to apply a long-term memory strategy.

2. A method as claimed in claim 1, wherein the control unit controls the user interface to initiate the request for the user to recall at least one rendered item that directly precedes the request at irregular or regular time intervals.

3. A method as claimed in claim 1, wherein the memory strategy applied by the user to the long-term memory training session provided by the user interface is selected, by the control unit (102), to be a working memory strategy in the event that the response received from the user to the request comprises an incorrect recall of the at least one rendered item that directly precedes the request.

4. A method as claimed in claim 1, wherein the memory strategy applied by the user to the long-term memory training session provided by the user interface is selected, by the control unit, to be a long-term memory strategy in the event that the response received from the user to the request comprises a correct recall of the at least one rendered item that directly precedes the request.

5. A method as claimed in claim 4, the method further comprising:
continuing the long-term memory training session provided by the user interface when the memory strategy applied by the user to the long-term memory training session provided by the user interface is selected, by the control unit, to be a long-term memory strategy.

6. (canceled)

7. A method as claimed in claim 6, wherein adapting, by the control unit, the long-term memory training session provided by the user interface comprises any one or more of:
controlling, by the control unit, the user interface to initiate a notification associated with a long-term memory strategy to the user to guide the user to apply a long-term memory strategy;
adding, by the control unit, at least one item to the one or more items rendered by the user interface to the user to guide the user to apply a long-term memory strategy;
controlling, by the control unit, the user interface to render a negative result associated with the working memory strategy to the user to guide the user to apply a long-term memory strategy; and
controlling, by the control unit, the user interface to instruct the user to re-start the long-term memory training session provided by the user interface to guide the user to apply a long-term memory strategy.

8. A method as claimed in claim 1, wherein the control unit controls the user interface to render two or more items in a predefined series.

9. A method as claimed in claim 1, wherein the control unit controls the user interface to render at least seven items to the user in the long-term memory training session provided by the user interface and the control unit controls the user interface to initiate the request for the user to recall at least one rendered item that directly precedes the request after rendering seven items to the user.

10. A computer program product comprising a computer readable medium, the computer readable medium having computer readable code embodied therein, the computer readable code being configured such that, on execution by a suitable computer or processor, the computer or processor is caused to perform the method of claim 9.

11. An apparatus for guiding a user in a long-term memory training session provided by a user interface, the apparatus comprising:
a control unit configured to:
control the user interface to render one or more items to the user in the long-term memory training session provided by the user interface;
control the user interface to initiate a request for the user to recall at least one rendered item that directly precedes the request;
select, from a plurality of memory strategies stored in a memory unit, a memory strategy applied by the user to the long-term memory training session provided by the user interface based on a response received from the user to the request; and
initiate an action to cause the user interface to guide the user to apply a long-term memory strategy to the long-term memory training session provided by the user interface in the event that the memory strategy applied by the user to the long-term memory training session provided by the user interface is selected to be a working memory strategy, wherein the control unit is configured to initiate an action to cause the user interface to guide the user to apply a long-term memory strategy by being configured to adapt the long-term memory training session provided by the user interface to guide the user to apply a long-term memory strategy.

12. An apparatus as claimed in claim 11, the apparatus further comprising the user interface.

13. An apparatus as claimed in claim 11, the apparatus further comprising a memory unit for storing one or more actions to guide the user to apply a long-term memory strategy to the long-term memory training session provided by the user interface.

14. An apparatus as claimed in claim 13, wherein the control unit is configured to acquire at least one action from the memory unit to initiate to guide the user to apply a long-term memory strategy to the long-term memory training session provided by the user interface.
